(19) United States
(12) Patent Application Publication
(10) Pub. No.: US 2016/0279322 A1
(43) Pub. Date: Sep. 29, 2016

(54) Dual chamber syringe with permeable diaphragm for forming emulsion

(71) Applicant: NEURON GUARD S.R.L.S., Modena (mo) (IT)

(72) Inventor: Enrico Giuliani, Modena (MO) (IT)

(73) Assignee: NEURON GUARD S.R.L., Modena (MO) (IT)

(21) Appl. No.: 14/777,928

(22) PCT Filed: Mar. 21, 2014
(86) PCT No.: PCT/IB2014/060032

§ 371 Date: Sep. 17, 2015

(30) Foreign Application Priority Data
Mar. 22, 2013 (IT) MO2013A000075

(51) Int. Cl. A61M 5/14; A61M 5/31; A61M 5/19; A61M 5/28; A61M 5/165; A61M 5/168

(52) U.S. Cl. CPC A61M 5/1409; A61M 5/165; A61M 5/16827; A61M 5/19; A61M 5/284; A61M 5/3145; A61M 2005/1657; A61M 2005/3121; A61M 2005/3128; A61M 2202/048; A61M 2202/08; A61M 2205/3306; A61M 2205/3334; A61M 2205/75; A61M 2205/7545

(57) ABSTRACT

An administration device includes a container body which defines therein a chamber containing at least two substances to be administered in at least two distinct, not spontaneously emulsifiable states; a permeable fragmentation diaphragm which divides the containing chamber into a first half-chamber in which substances to be administered are loaded and a contiguous second half-chamber in which the at least two substances to be administered are mixed; a thrust member pushing at least one of the at least two substances to be administered from the first half-chamber into second half-chamber through the permeable diaphragm and pushing the at least two substances after emulsification from the second semi-chamber into a recipient; and an outlet from the second half-chamber to the recipient.

### FIELD OF THE INVENTION

The invention relates to an administration device, which can be generally used for administering drugs and drug mixtures to patients, particularly for administering metered doses of halogenated anesthetic agents to patients who suffered hypoxic or anoxic neuronal injury.

### BACKGROUND ART

It has been known for some time that patients who suffered hypoxic or anoxic brain injury after surgery or trauma may be treated with sedative drugs to decrease neuronal metabolism and, as a result, uncontrolled expansion of tissue damage, thereby limiting the occurrence of invalidating residual disabilities.

Particularly, US2004/0127578 discloses a method for cardioprotection and neuroprotection by intravenous administration of halogenated volatile anesthetics.

Typically, this patent provides a method for treating a patient having a tissue that is subject to an ischemic event.

The method is conducted by parenterally administering a formulation containing a halogenated volatile anesthetic in an amount effective to improve the tissue's resistance to or tolerance of the ischemic event

In the preferred embodiment of the invention, the amount of the formulation administered to the patient is a sub-anesthetic.

The formulation may be administered prior to, concurrently with, or after the ischemic event.

The method may be used, for instance, for treatment of patients having a myocardial or neuronal tissue that is subject to an ischemic event.

Nevertheless, this prior art suffers from certain drawbacks.

A first drawback is that the administered formulations comprise lipid solutions that have preservation problems, as they can be aggressed by contaminating pathogens.

Another drawback is that these lipid solutions have storage problems and a limited range of administration temperatures.

A further drawback is that these lipid solutions may induce allergenicity in particularly predisposed patients.

Yet another drawback is that continuous intravenous administration of a lipid solution throughout the treatment period may expose the body of the patient to a detrimental overload, and cause liver and lung alterations that would increase cardiovascular hazard.

A further drawback is that patients that have undergone an ischemic event are generally required to be treated in a hospital, whereby the suffering patient must be first moved from the place in which the event occurred, which will cause the loss of precious time to implement the required therapies and limit propagation of neuronal injury, which progressively necrotizes cells as time passes, due to neuronal hyperactivity induced thereby.

### DISCLOSURE OF THE INVENTION

The invention has the object to improve the prior art.

Another object of the invention is to provide an administration device that can be generally used for administering drugs and drug mixtures to patients, particularly for administering metered doses of halogenated anesthetic agents to patients who suffered hypoxic or anoxic neuronal injury, and allows effective treatment of patients in the place where an ischemic event has occurred.

A further object of the invention is to provide an administration device that allows administration of metered doses of anesthetic agents to patients either in quick, “bolus” form, i.e. with short administration times, typically five minutes or less, or as a continuous infusion, with the doses of anesthetic agents being allowed to be continuously metered according to the particular conditions of the patients.

In one aspect, the invention provides an administration device as defined by the features of claim 1.

In another aspect, the invention provides an administration method as defined by the features of claim 12.

The invention achieves the following advantages:

prompt administration of metered doses of halogenated anesthetic agents to patients that have suffered an ischemic event, to decrease neuronal activity in the brain and limit the occurrence of irreversible tissue injury;

continuous metering of administered doses according to the conditions of the patients being treated;

continuous parenteral administration of halogenated anesthetic agents.

### BRIEF DESCRIPTION OF THE DRAWINGS

Further features and advantages of the invention will be more readily apparent upon reading of the detailed description of preferred non-exclusive embodiments of an administration device, which is shown as a non-limiting example in the annexed drawings, in which:

FIG. 1 is a perspective view of a first possible embodiment of an administration device of the invention, at the start of administration;

FIGS. 3 to 5 are schematic longitudinal sectional views of the administering device of FIG. 1, during administration;

FIG. 6 is a detail view of a reference member that is adapted to be removably mounted to the administration device;

FIG. 7 is a cross-sectional view of the reference member of FIG. 6, as taken along a plane VI - VI;

FIG. 8 is a schematic view of a second possible embodiment of the administration device of the invention;

FIG. 9 is a smaller-scale perspective view of the administration device of FIG. 8.

### DETAILED DESCRIPTION OF A PREFERRED EMBODIMENT

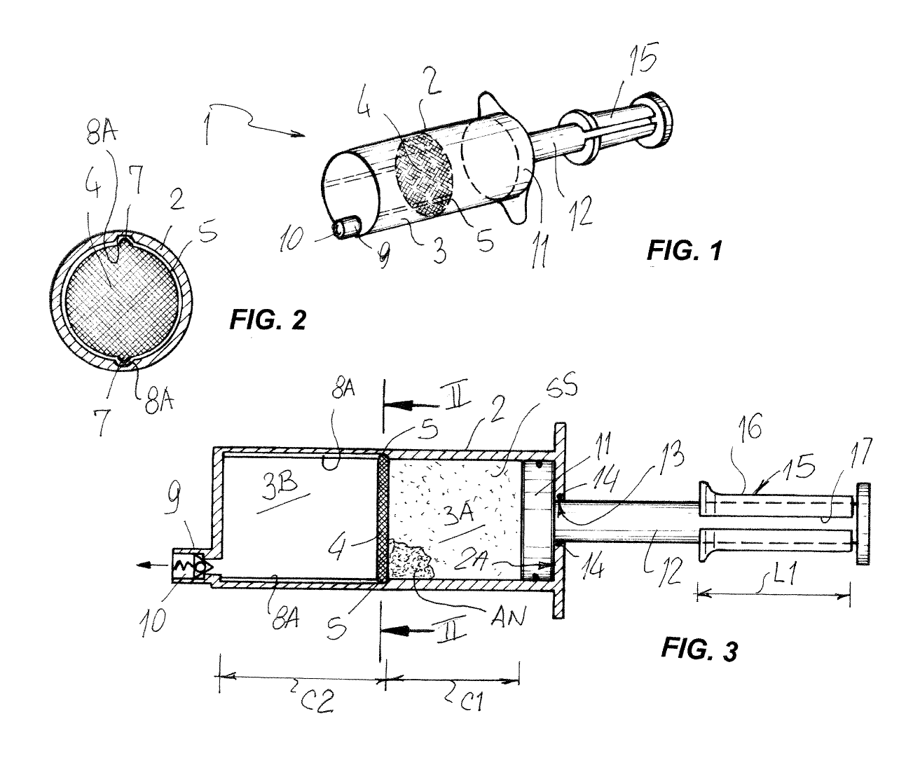

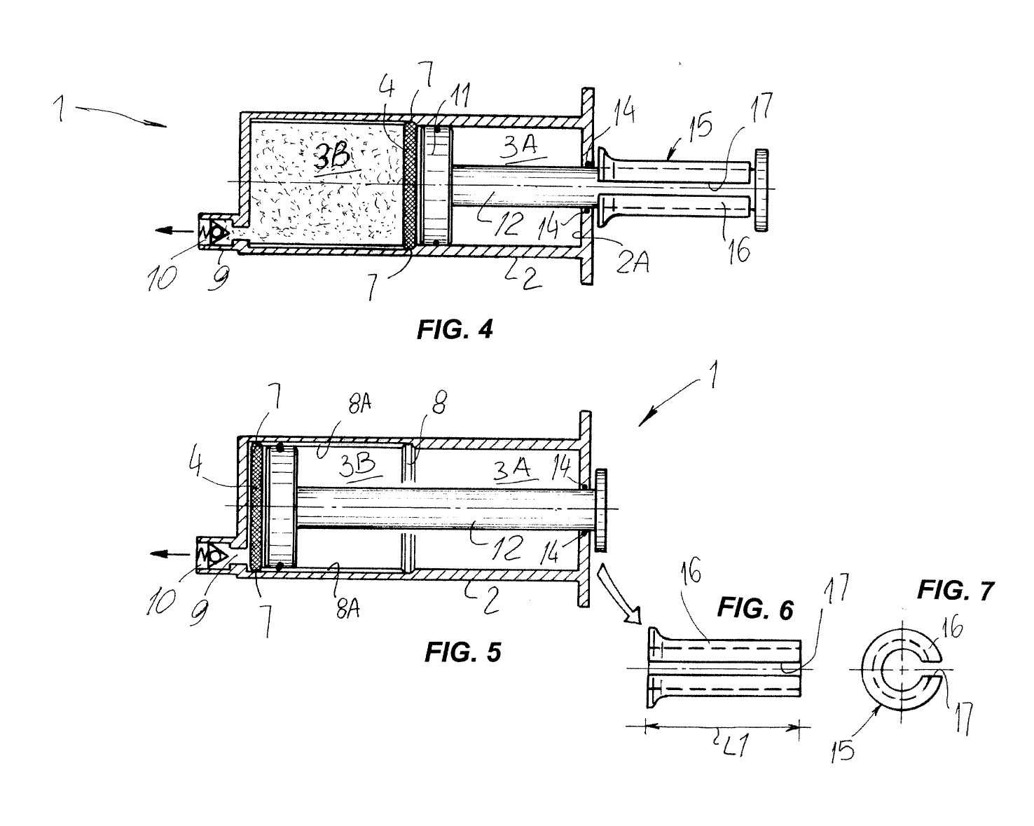

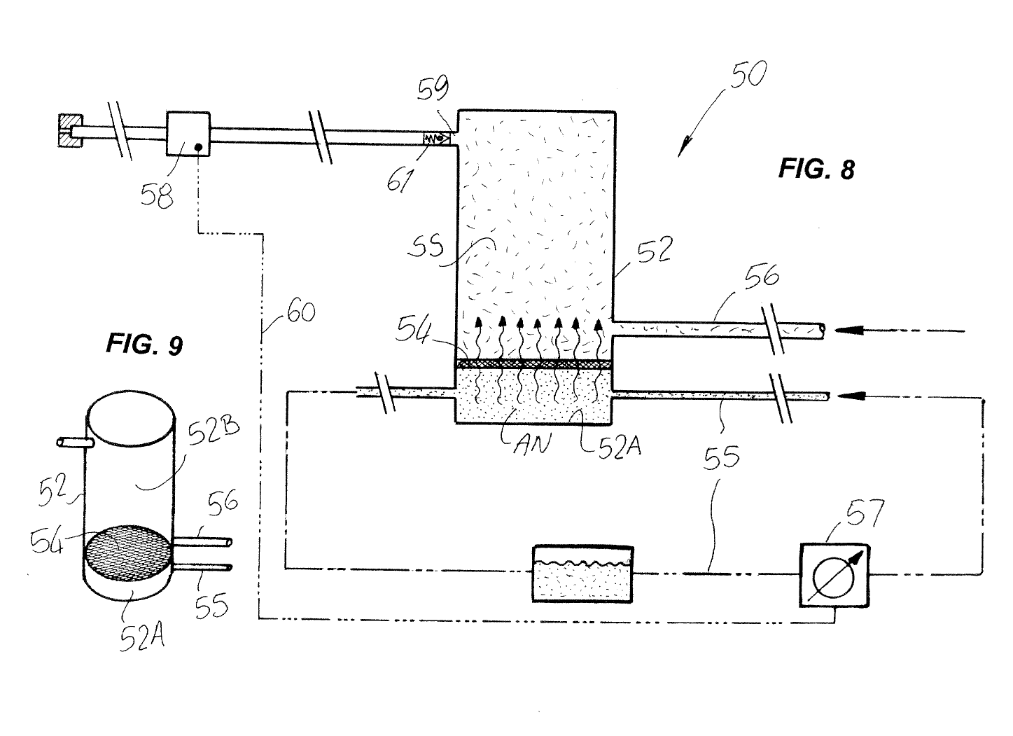

Referring now to FIGS. 1 to 5, numeral 1 generally designates an administration device according to a first embodiment, particularly but without limitation adapted for “bolus” administration for quick administration and, as needed, in the place where an ischemic event occurs.

In this embodiment, the device 1 is provided in the form of a syringe which typically comprises a container body 2 with a containing chamber 3 defined therein.

A microporous and hence permeable diaphragm 4 is transversely mounted in this chamber 3 using support means.

The diaphragm 4 comprises a stretching and support frame 5 which circumscribes the inner microporous part and is removably mounted in the containing chamber 3 using coupling and support means.

The latter comprise a raised peripheral rib 7, which extends from the frame 5 and is designed to be interlockingly received in a corresponding concave annular seat 8 formed in the containing chamber 3, and a pair of parallel longitudinal guides 8A arranged in the emulsification half-chamber 3B.

In practice, the diaphragm 4 divides the containing chamber into two half-chambers, i.e. a first loading half-chamber 3A and a contiguous second emulsification half-chamber 3B.

Furthermore, the second half-chamber 3B has an outlet 9 for the emulsified substances to be administered to a patient, which is equipped with a non-return valve 10 that prevents backflow of the substances into the second emulsification chamber 3B.

In a pre-administration condition, the first loading chamber 3A slidably and sealingly houses a plunger 11 having a shaft that coaxially extends out of the container chamber 3 through an opening 13 formed in a proximal wall 2A of the body 2 and having seals 14.

A removable reference member 15 is temporarily mounted to the stem 12, which has the purpose of pushing the plunger 11 into the container chamber toward the outlet 9, and its length, referenced “L1” in the figures is substantially equal to the length of a first stroke “C1” that the plunger 11 has to run to move from the proximal wall 2A to contact with the diaphragm 4.

The reference member 15 is actually a sleeve 16 with a longitudinal slit extending throughout its length “L1”, which allows it to elastically open apart to a sufficient extent as to allow it to be fitted onto the shaft 12 or be removed therefrom.

In the first loading chamber 3A two substances to be administered to a patient that has suffered an ischemic event are loaded, for decreasing neuronal injury in the brain.

More in detail, these two substances comprise a halogenated and volatile anesthetic agent, schematically referenced “AN”, and a solvent solution “SS”, which are separate from each other, and they are originally in two different, non-emulsifiable states.

The diaphragm 4 may be formed either as a filter or as a membrane, but in both cases it is of microporous nature, for both substances to pass therethrough when the plunger 11 is pushed by the shaft 12 actuated by a medical operator, as better explained hereinafter.

In the second embodiment of the administration device, which is particularly suitable for sustained administration, instead of “bolus” administration, the administration device of the invention is referenced 50 and still comprises a body 52 defining therein a first loading half-chamber 52A and a contiguous second emulsification half-chamber 52B.

The two half-chambers are also divided by a microporous diaphragm 54, which is very similar to the diaphragm 4 as used in the previously described embodiment of the administration device 1.

A feeding circuit 55 is connected to the first half-chamber 52A for feeding the halogenated and volatile anesthetic agent “AN”, and a feeding line 56 is connected to the second half-chamber 52B for feeding the solvent solution “SS”.

An adjustable-flow pump 57 is also mounted to the circuit 55, to maintain the anesthetic agent “AN” in circulation, and create a pressure in the first half-chamber 52A, which is higher than the pressure in the second half-chamber 52B, to push the anesthetic agent “AN” through the diaphragm 54.

Like in the previously described embodiment, the second half-chamber 52B has an outlet 59 through which the emulsified substances may reach a patient that is designed to be treated thereby.

The outlet 59 has both the non-return valve 61 and a detection device 58 for detecting the concentration of the anesthetic agent “AN” in the solvent solution “SS”, mounted thereto.

This detection device 58 is typically known to the skilled person and may be provided, for instance, in the form of an optical sensor, which is connected to the pump 57 via a connection line 60, to automatically control flow according to current detections, as constantly compared with the desired and pre-settable concentration values.

The operation of the administration device in the first embodiment of the invention is as follows: a dose of the anesthetic agent “AN” and a dose of the solvent solution “SS” are loaded into the first loading half-chamber 3A in which they remain in separate form, due to their different, not spontaneously emulsifiable states.

The medical operator fits the reference member 15 onto the shaft 12, if this has not been done at the start, and opens it apart enough to straddle the shaft and coaxially surround it in loose fashion.

For administration of the doses of the two substances in the first half-chamber 3A, the operator pushes the shaft 12 and causes the plunger 11 to contact the diaphragm 4 through a first stroke “C1”.

During this first stroke “C1”, both the dose of the anesthetic agent “AN” and the dose of the solvent solution “SS” pass through the micropores of the diaphragm 4 and the anesthetic agent “AN”, which is typically of lipid nature, breaks up into microparticles that move into the second emulsification half-chamber 3B with the solvent solution “SS”, and emulsify therewith due to their crushed state.

In this configuration, the reference member 15 rests upon the outer face of the proximal wall 2A to inform the medical operator that both substances have been pushed into the emulsification chamber 3B.

Therefore, the medical operator will remove the reference member 15 and push the shaft 12 again.

The plunger will push the diaphragm 4 and remove it from the concave annular seat 8 to progressively guide it toward the outlet 9 and also push both emulsified substances therethrough toward the receiving patient.

The one-way valve 10 prevents any backflow of both emulsified substances during administration.

In the second embodiment of the administration device 50, the pushing action that forces the anesthetic agent “AN” to pass through the diaphragm 54 that breaks it up into micro-particles is generated by the pressure created by the pump 57 that is mounted to the feeding circuit 55.

More in detail, the pump 57 keeps the anesthetic agent “AN” circulating in the circuit 55, while the solvent solution “SS” is fed into the second half-chamber 52B.

The overpressure created in the first half-chamber 52A generates a thrust that forces the anesthetic agent “AN” to pass through the microporous diaphragm 54 and break up into micro-particles that flow into the second half-chamber 52B and emulsify with the solvent solution “SS”.

When both substances are emulsified, they are pushed toward the outlet 59, through which they reach the receiving patient.

As they pass through the outlet 58, the detection device 58 detects the concentration of the micro-particles of anesthetic agent “AN” in the solvent solution “SS” and compares it with a desired value that has been preset by the medical operator.

If the detected value does not match the preset value, then the detection device will control the pump 57 through the connection line 60 to change its flow for the concentration of the anesthetic agent “AN” to match the preset value.

The invention has been found to fulfill the intended objects.

The invention so conceived is susceptible to changes and variants within the inventive concept.

Furthermore, all the details may be replaced by other technically equivalent parts.

In practice, any material, shape and size may be used as needed, without departure from the scope as defined by the following claims.

The invention claimed is:

1. An administration device comprising:
a container body (2; 52) which defines in an inside thereof a containing chamber (3) of at least two substances (AN, SS) to be administrated in at least two different states which cannot be mixed spontaneously;
a fragmentation permeable diaphragm (4; 54) which divides said containing chamber (3) into a first loading semi-chamber (3A; 52A) of said at least two substances (AN, SS) to be administrated and ad adjacent second mixing semi-chamber (3B; 52B) of said at least two substances to be administrated;
a pushing member (11, 12) of at least one of said at least two substances to be administrated from said first semi-chamber (3A; 52A) into said second semi-chamber (3B; 52B) through said permeable diaphragm (4; 54) and of said at least two substances (AN, SS) already mixed from said second semi-chamber (3B; 52B) to an administration addressee; and
one outlet (9; 59) from said second semi-chamber (3B; 52B) toward said administration addressee.

2. The administration device as claimed in claim 1, wherein said outlet (9; 59) comprises a concentration sensing device (58) of at least one of said substances to be administrated.

3. The administration device as claimed in claim 1, wherein said permeable diaphragm (4; 54) is a semi-permeable membrane or a micro-porous filter.

4. The administration device as claimed in claim 1, wherein said diaphragm (4; 54) is movably supported in said containing chamber (3) by support guides (8).

5. The administration device as claimed in claim 4, wherein said permeable diaphragm (4; 54) is peripherally equipped with a stretching and supporting frame (5), movably coupled with said support guides (8) by coupling means (7).

6. The administration device as claimed in claim 5, wherein said support guides comprise:
an concave annular seat (8) transversally obtained inside said containing chamber (3), wherein said coupling means (7) are designed to be engaged in a removable way; and
a couple of parallel grooves (8A) extending from said annular seat (8) toward said outlet (9; 59), wherein said coupling means (7) are slidingly received.

7. The administration device as claimed in claim 1, wherein said pushing member comprises a plunger (11) slidingly housed in said containing chamber (3) and movable between said first semi-chamber (3A; 52A) and second semi-chamber (3B; 52B) along a first movement stroke (C1) and a subsequent second movement stroke (C2), said plunger having a push stem (12) sealingly prolonging out of said containing chamber (3).

8. The administration device as claimed in claim 7, wherein said push stem (12) has a length at least equal to a sum of said first movement stroke (C1) and second movement stroke (C2).

9. The administration device as claimed in claim 7, wherein a reference element (15) having a length equal to said first movement stroke (C1) is placed on said push stem (12).

10. The administration device as claimed in claim 1, wherein said pushing member comprises a feeding circuit (55) of at least one of said substances to be administrated that flows into said first semi-chamber (3A; 52A).

11. The administration device as claimed in claim 10, wherein said feeding circuit comprises a pressurized circuit (55) equipped with a pump (57) designed to create a pressure inside said first semi-chamber (3A; 52A) greater than a pressure inside said second semi-chamber (3B; 52B).

12. An administration method of at least a first and a second substance (AN, SS) to be administered in at least two different states which cannot be mixed spontaneously, comprising:
loading a first substance (AN) to be administrated in a first loading semi-chamber (3A; 52A) of a containing chamber (3) defined inside an administration device (1; 50) having a second mixing semi-chamber (3B; 52B) adjacent to said first semi-chamber (3A; 52A);
pushing said first substance (AN) through a permeable fragmentation diaphragm (4; 54) placed between said first (3A; 52A) and said second semi-chamber (3B; 52B), so that said first substance fragments into micro-particles;
mixing said micro-particles into said second substance (SS), and
administering said first and said second substances (AN, SS) mixed together to an administration addressee.
